(19) United States
(12) Patent Application Publication
(10) Pub. No.: US 2003/0162815 A1
(43) Pub. Date: Aug. 28, 2003

(54) Thiazolidinedione salt for treatment of diabetes mellitus

(76) Inventors: Andrew Simon Craig, Kent (GB); Tim Chien Ting Ho, Kent (GB)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/297,651

(22) PCT Filed: Jun. 8, 2001
(86) PCT No.: PCT/GB01/02567

(51) Int. Cl. A61K 31/4439; C07D 417/02

(52) U.S. Cl. 514/342; 424/9.3; 546/269.7

(57) ABSTRACT

A novel pharmaceutical compound 5-[4-[2-(N-methyl-N-(2-pyridyl)amino)ethoxy]benzyl]thiazolidine-2,4-dione hydrobromide or a solvate thereof, a process for preparing such a compound, a pharmaceutical composition comprising such a compound and the use of such a compound in medicine.

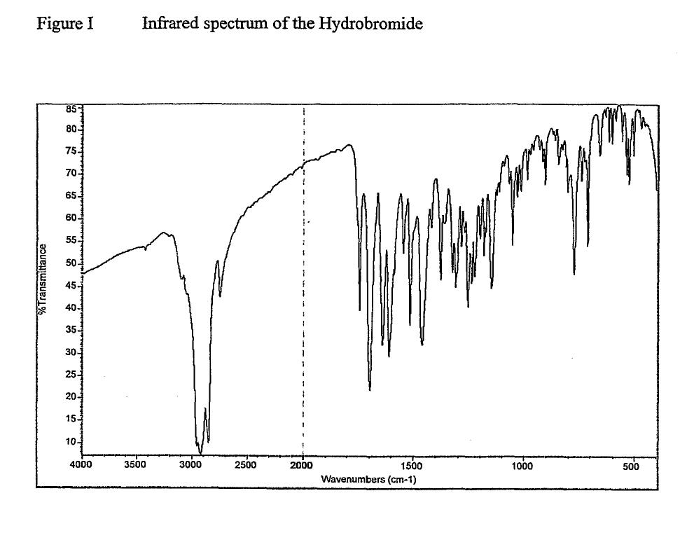

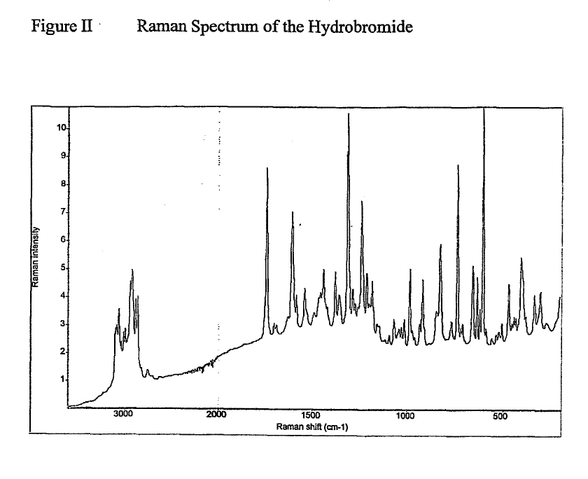

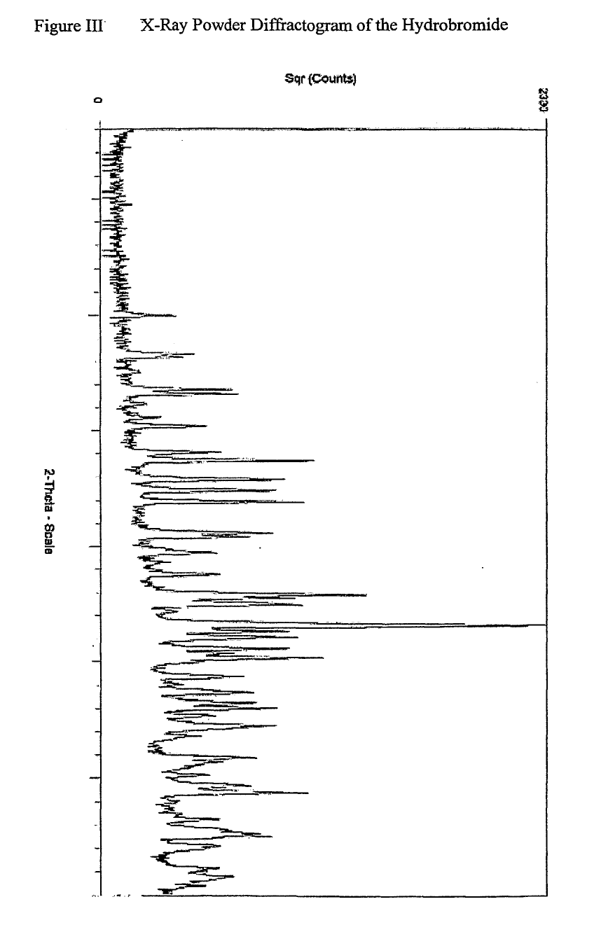

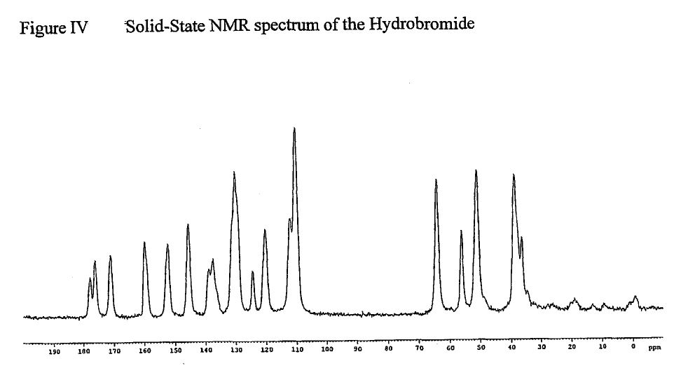

This invention relates to a novel pharmaceutical, to a process for the preparation of the pharmaceutical and to the use of the pharmaceutical in medicine.

European Patent Application, Publication Number 0,306,228 relates to certain thiazolidinedione derivatives disclosed as having hypoglycaemic and hypolipidaemic activity. The compound of example 30 of EP 0,306,228 is 5-[4-[2-(N-methyl-N-(2-pyridyl)amino)ethoxy]benzyl]thiazolidine-2,4-dione (hereinafter also referred to as “Compound (I)”).

International Patent Application, Publication Number WO94/05659 discloses certain salts of the compounds of EP 0,306,228 including salts formed from mineral acids such as hydrobromic, hydrochloric and sulphuric acids, and organic acids, such as methanesulphonic, tartaric and, in-particular, maleic acid salts.

It has now been discovered that Compound (I) forms a novel hydrobromide salt (hereinafter also referred to as the “Hydrobromide”) that is particularly stable and hence is suitable for bulk preparation and handling. The Hydrobromide also has a high melting point, shows particularly good aqueous solubility and possesses good bulk flow properties. The hydrobromide is therefore surprisingly amenable to large scale pharmaceutical processing and especially to large scale riling.

The novel form can be prepared by an efficient, economic and reproducible process particularly suited to large-scale preparation.

The novel Hydrobromide also has useful pharmaceutical properties and in particular it is indicated to be useful for the treatment and/or prophylaxis of diabetes mellitus, conditions associated with diabetes mellitus and certain complications thereof.

Accordingly, the present invention provides 5-[4-[2-(N-methyl-N-(2-pyridyl)amino)ethoxy]benzyl]thiazolidine-2,4-dione hydrobromide or a solvate thereof.

Suitably, the hydrobromide is a monohydrobromide.

In one favoured aspect, the Hydrobromide provides an infrared spectrum substantially in accordance with FIG. I.

In one favoured aspect, the Hydrobromide provides a Raman spectrum substantially in accordance with FIG. II.

In one favoured aspect, the Hydrobromide provides an X-Ray powder diffraction pattern (XRPD) substantially in accordance with Table I or Figure III.

In one favoured aspect, the Hydrobromide provides a solid-state ¹³C NMR spectrum substantially in accordance with FIG. IV.

It is also favoured that the Hydrobromide has a melting point within the range of from 175 to 185° C., especially 180 to 185° C., for example 181° C.

Also the Hydrobromide has a Tₒₙₛₑₜ within the range of from 180 to 186° C., for example 182.5° C.

Thus in a preferred aspect, the the Hydrobromide is characterised in that it provides two or more of:

(i) an infrared spectrum substantially in accordance with FIG. I;

(ii) a Raman spectrum substantially in accordance with FIG. II;

(iii) an X-Ray powder diffraction pattern (XRPD) substantially in accordance with Table I or FIG. III;

(iv) a solid-state ¹³C NMR spectrum substantially in accordance with FIG. IV; and

(v) a melting point within the range of from 175 to 185° C., especially 180 to 185° C., for example 181° C.

The present invention encompasses the Hydrobromide or solvate thereof isolated in pure form or when admixed with other materials. Thus in one aspect there is provided the Hydrobromide or solvate thereof in isolated form.

In a further aspect there is provided the Hydrobromide or solvate thereof in a purified form.

In yet a further aspect there is provided the Hydrobromide or solvate thereof in crystalline form.

Also, the invention provides the Hydrobromide or solvate thereof in a solid pharmaceutically acceptable form, such as a solid dosage form, especially when adapted for oral administration.

Moreover, the invention also provides the Hydrobromide or solvate thereof in a pharmaceutically acceptable form, especially in bulk form, such form being particularly capable of being milled.

Furthermore, the invention provides the Hydrobromide or solvate thereof in a pharmaceutically acceptable form, especially in bulk form, such form having good flow properties, especially good bulk flow properties.

A suitable solvate is a hydrate.

The invention also provides a process for preparing the Hydrobromide or solvate thereof, characterised in that 5-[4-[2-(N-methyl-N-(2-pyridyl)amino)ethoxy]benzyl]thiazolidine-2,4-dione (Compound(I)), or a salt thereof, preferably dispersed or dissolved in a suitable solvent, is reacted with a source of hydrogen bromide and thereafter, if required, a solvate of the Hydrobromide is prepared; and the Hydrobromide or solvate thereof is recovered.

A suitable reaction solvent is an alkanol, for example propan-2-ol, or a hydrocarbon, such as toluene, a ketone, such as acetone, an ester, such as ethyl acetate, an ether such as tetrahydrofuran, a nitrile such as acetonitrile, or a halogenated hydrocarbon such as dichloromethane, water, or an organic acid such as acetic acid; or a mixture thereof.

Conveniently, the source of hydrogen bromide is provided by an aqueous solution of hydrogen bromide, for example a 48% w/w solution in water. Alternatively, the source of hydrogen bromide is a solution of hydrogen bromide in an appropriate solvent, optionally the reaction solvent, for example propan-2-ol. In addition, the hydrogen bromide may be added directly to a solution or suspension of Compound(I) in the chosen reaction solvent.

An alternative source of hydrogen bromide is provided by a base salt of hydrobromic acid for example ammonium bromide, or the hydrobromic acid salt of an amine, for example ethylamine or diethylarnine.

The reaction is usually carried out at ambient temperature or at an elevated temperature, for example at the reflux temperature of the solvent, although any convenient temperature that provides the required product may be employed.

Solvates, such as hydrates, of the Hydrobromide are prepared according to conventional procedures.

Recovery of the required compound generally comprises crystallisation from an appropriate solvent, conveniently the reaction solvent, usually by cooling to a temperature in the range of from 0° C. to 60° C., for example 20 to 25° C or from 40 to 50° C. For example the Hydrobromide may be crystallised from an alcohol such as propan-2-ol or a ketone such as acetone.

In one preferred form the recovery comprises initial cooling to a first temperature, such as in the range of from 40 to 50° C., thereby allowing initiating crystallisation and thereafter cooling to a second temperature, suitably in the range of from 0 to 25° C.

Crystallisation can also be initiated by seeding with crystals of the Hydrobromide or solvate thereof but this is not essential.

Solvates are prepared by use of appropriate conventional methods.

Compound (I) is prepared according to known procedures, such as those disclosed in EP 0,306,228 and WO94/05659. The disclosures of EP 0,306,228 and WO94/05659 are incorporated herein by reference.

When used herein the term “Tₒₙₛₑₜ” is generally determined by Differential Scanning Calorinetry and has a meaning generally understood in the art, as for example expressed in Pharmaceutical Thermal Analysis, Techniques and Applications”, Ford and Timmins, 1989 as “The temperature corresponding to the intersection of the pre-transition baseline with the extrapolated leading edge of the transition”.

When used herein in respect of certain compounds the term “good flow properties” is suitably characterised by the said compound having a Hausner ratio of less than or equal to 1.5, especially of less than or equal to 1.25.

“Hausner ratio” is an art accepted term.

When used herein the term ‘prophylaxis of conditions associated with diabetes mellitus’ includes the treatment of conditions such as insulin resistance, impaired glucose tolerance, hyperinsulinaemia and gestational diabetes.

Diabetes mellitus preferably means Type II diabetes mellitus.

Conditions associated with diabetes include hyperglycaemia and insulin resistance and obesity. Further conditions associated with diabetes include hypertension, cardiovascular disease, especially atherosclerosis, certain eating disorders, in particular the regulation of appetite and food intake in subjects suffering from disorders associated with under-eating, such as anorexia nervosa, and disorders associated with over-eating, such as obesity and anorexia bulimia. Additional conditions associated with diabetes include polycystic ovarian syndrome and steroid induced insulin resistance.

The complications of conditions associated with diabetes mellitus encompassed herein includes renal disease, especially renal disease associated with the development of Type II diabetes including diabetic nephropathy, glomerulonephritis, glomerular sclerosis, nephrotic syndrome, hypertensive nephrosclerosis and end stage renal disease.

As mentioned above the compound of the invention has useful therapeutic properties: The present invention accordingly provides the Hydrobromide or solvate thereof for use as an active therapeutic substance.

More particularly, the present invention provides the Hydrobromide or solvate thereof for use in the treatment and/or prophylaxis of diabetes mellitus, conditions associated with diabetes mellitus and certain complications thereof.

The Hydrobromide or solvate thereof may be administered per se or, preferably, as a pharmaceutical composition also comprising a pharmaceutically acceptable carrier. Suitable methods for formulating the Hydrobromide or solvate thereof are generally those disclosed for Compound (I) in the above mentioned publications.

Accordingly, the present invention also provides a pharmaceutical composition comprising the Hydrobromide or solvate thereof and a pharmaceutically acceptable carrier therefor.

The Hydrobromide or solvate thereof is normally administered in unit dosage form.

The active compound may be administered by any suitable route but usually by the oral or parenteral routes. For such use, the compound will normally be employed in the form of a pharmaceutical composition in association with a pharmaceutical carrier, diluent and/or excipient, although the exact form of the composition will naturally depend on the mode of administration.

Compositions are prepared by admixture and are suitably adapted for oral, parenteral or topical administration, and as such may be in the form of tablets, capsules, oral liquid preparations, powders, granules, lozenges, pastilles, reconstitutable powders, injectable and infusable solutions or suspensions, suppositories and transdermal devices. Orally administrable compositions are preferred, in particular shaped oral compositions, since they are more convenient for general use.

Tablets and capsules for oral administration are usually presented in a unit dose, and contain conventional excipients such as binding agents, fillers, diluents, tabletting agents, lubricants, disintegrants, colourants, flavourings, and wetting agents. The tablets may be coated according to well known methods in the art.

Suitable fillers for use include cellulose, mannitol, lactose and other similar agents. Suitable disintegrants include starch, polyvinylpyrrolidone and starch derivatives such as sodium starch glycollate. Suitable lubricants include, for example, magnesium stearate. Suitable pharmaceutically acceptable wetting agents include sodium lauryl sulphate.

Solid oral compositions may be prepared by conventional methods of blending, filling, tabletting or the like. Repeated blending operations may be used to distribute the active agent throughout those compositions employing large quantities of fillers. Such operations are, of course, conventional in the art.

Oral liquid preparations may be in the form of, for example, aqueous or oily suspensions, solutions, emulsions, syrups, or elixirs, or may be presented as a dry product for reconstitution with water or other suitable vehicle before use. Such liquid preparations may contain conventional additives such as suspending agents, for example sorbitol, syrup, methyl cellulose, gelatin, hydroxyethylcellulose, carboxymethyl cellulose, aluminum stearate gel or hydrogenated edible fats, emulsifying agents, for example lecithin, sorbitan monooleate, or acacia; non-aqueous vehicles (which may include edible oils), for example, almond oil, fractionated coconut oil, oily esters such as esters of glycerine, propylene glycol, or ethyl alcohol; preservatives, for example methyl or propyl p-hydroxybenzoate or sorbic acid, and if desired conventional flavouring or colouring agents.

For parenteral administration, fluid unit dose forms are prepared containing a compound of the present invention and a sterile vehicle. The compound, depending on the vehicle and the concentration, can be either suspended or dissolved. Parenteral solutions are normally prepared by dissolving the active compound in a vehicle and filter sterilising before filling into a suitable vial or ampoule and sealing. Advantageously, adjuvants such as a local anaesthetic, preservatives and buffering agents are also dissolved in the vehicle. To enhance the stability, the composition can be frozen after filling into the vial and the water removed under vacuum.

Parenteral suspensions are prepared in substantially the same manner except that the active compound is suspended in the vehicle instead of being dissolved and sterilised by exposure to ethylene oxide before suspending in the sterile vehicle. Advantageously, a surfactant or wetting agent is included in the composition to facilitate uniform distribution of the active compound.

As is common practice, the compositions will usually be accompanied by written or printed directions for use in the medical treatment concerned.

As used herein the term ‘pharmaceutically acceptable’ embraces compounds, compositions and ingredients for both human and veterinary use: for example the term ‘pharmaceutically acceptable salt’ embraces a veterinarily acceptable salt.

The present invention further provides a method for the treatment and/or prophylaxis of diabetes mellitus, conditions associated with diabetes mellitus and certain complications thereof, in a human or non-human mammal which comprises administering an effective, non-toxic, amount of Hydrobromide or solvate thereof to a human or non-human mammal in need thereof.

Conveniently, the active ingredient may be administered as a pharmaceutical composition hereinbefore defined, and this forms a particular aspect of the present invention.

In a further aspect the present invention provides the use of Hydrobromide or solvate thereof for the manufacture of a medicament for the treatment and/or prophylaxis of diabetes mellitus, conditions associated with diabetes mellitus and certain complications thereof.

In the treatment and/or prophylaxis of diabetes mellitus, conditions associated with diabetes mellitus and certain complications thereof the Hydrobromide or solvate thereof may be taken in amounts so as to provide Compound (I) in suitable doses, such as those disclosed in EP 0,306,228, WO94/05659 or WO98/55 122.

No adverse toxicological effects are indicated in the above mentioned treatments for the compounds of the invention.

The following examples illustrate the invention but do not limit it in any way.

### EXAMPLE 1

5-[4-[2-(N-methyl-N-(2-pyridyl)amino)ethoxy]benzyl]thiazolidine-2,4-dione hydrobromide

A mixture of 5-[4-[2-(N-methyl-N-(2-pyridyl)amino)ethoxy]benzyl]thiazolidine-2,4-dione (1.0 g) and propan-2-ol (50 ml) was stirred and heated to reflux for a period of 10 minutes at which point a clear solution was observed. Hydrobromic acid (48 % w/w solution in water, 0.31 ml) was then added dropwise, and the reaction mixture stirred for 10 minutes at reflux then allowed to cool to 21° C. The product was collected by filtration and washed with propan-2-ol (10 ml) to give the 5-[4-[2-(N-methyl-N-(2-pyridyl)amino)ethoxy]benzyl]thiazolidine-2,4-dione hydrobromide (0.41 g) as a white crystalline solid.

### EXAMPLE 2

5-[4-[2-(N-methyl-N-(2-pyridyl)amino)ethoxy]benzyl]thiazolidine-2,4-dione hydrobromide

A mixture of 5-[4-[2-(N-methyl-N-(2-pyridyl)amino)ethoxy]benzyl]thiazolidine-2,4-dione (3.0 g) and acetone (100 ml) was stirred and heated to reflux for a period of 15 minutes at which point a clear solution was observed.

Hydrobromic acid (48% w/w solution in water, 0.95 ml ) was added and the reaction mixture stirred at reflux for 15 minutes and then cooled to 21° C. After standing for 120 hours the mother liquor was decanted and the crystalline product washed with acetone (10 ml) and dried under vacuum for 3 hours to give 5-[4-[2-(N-methyl-N-(2-pyridyl)amino)ethoxy]benzyl]thiazolidine-2,4-dionehydrobromide (3.7 g).

1H-NMR (d6-DMSO): consistent with 5-[4-[2-(N-methyl-N-(2-pyridyl)amino)ethoxy]benzyl]thiazolidine-2,4-dione hydrobromide containing acetone, 0.5% wt/wt.

### EXAMPLE 3

5-[4-[2-(N-methyl-N-(2-pyridyl)amino)ethoxy]benzyl]thiazolidine-2,4-dione hydrobromide

A mixture of 5-[4-[2-(N-methyl-N-(2-pyridyl)amino)ethoxy]benzyl]thiazolidine-2,4-dione (15.0 g) and acetone (230 ml) was stirred and heated to reflux for 15 minutes at which point a clear solution was observed. Hydrobromic acid (48% w/w aqueous solution, 4.75 ml) was then added and the mixture was cooled to 45° C. and stirred for 1 hour, and then cooled to 21° C. The white solid was collected by filtration, and washed with acetone (100 ml) to give 5-[4-[2-(N-methyl-N-(2-pyridyl)amino)ethoxy]benzyl]thiazolidine-2,4-dione hydrobromide (17.7 g) as a white crystalline solid.

Characterising Data for the Hydrkobromide

(1) Recorded for the Product of Example 3

Solubility of the Hydrobromide

The solubility of the material was determined by adding water in aliquots from 1 to 100 ml to approximately 100 mg of drug substance until the powder had dissolved. The visual solubility was confirmed by an HPLC assay of a saturated solution.

Solubility: 6 mg/ml.

Solid State Stability of the Hydrobromide

The solid state stability of the drug substance was determined by storing approximately 1.0 g of the material in a glass bottle at i) 40° C./75% Relative Humidity (RH), open exposure, for 1 month and b) at 50° C., closed, for 1 month. The material was assayed by HPLC for final content and degradation products in both cases.

a) 40° C / 75% RH: No significant degradation observed (HPLC assay 98% initial).

b) 50° C.: No significant degradation observed (HPLC assay 98% initial).

Flow Properties of the Hydrobromide:

The ratio between the bulk density and the tapped bulk density (Hausner Ratio) of the Hydrobromide was determined using standard methods (“Pharmaceutics—The Science of Dosage Form Design”, editor M. Aulton, 1988, published by: Churchill Livingstone).

Hausner Ratio: 1.3

Tₒₙₛₑₜ of the Hydrobromide

The Tₒₙₛₑₜ of the drug substance was determined by Differential Scanning Calorimetry using a Perkin-Elmer DSC7 apparatus.

Tₒₙₛₑₜ: 182.5° C.

Melting Point of the Hydrobromide

The melting point of the drug substance was determined visually by hot stage microscopy.

Mpt: 181° C.

(2) Recorded for the Product of Example 2

The infrared absorption spectrum of a mineral oil dispersion of the product was obtained using a Nicolet 710 FT-IR spectrometer at 2 cm⁻¹ resolution (FIG. I). Data were digitised at 1 cm⁻¹ intervals. Bands were observed at:

2923, 2854, 2749, 1745, 1698, 1643, 1610, 1544, 1515, 1459, 1419, 1378, 1327, 1313, 1287, 1256, 1240, 1228, 1203, 1185, 1151, 1071, 1054, 1032, 1014, 985, 906, 803, 771, 738, 712, 524 cm⁻¹.

The IR spectrum of the solid product was recorded using a universal ATR accessory. Bands were observed at: 2929, 2859, 2749, 1745, 1694, 1641, 1608, 1543, 1514, 1445, 1419, 1382, 1358, 1326, 1311, 1287, 1255, 1240, 1202, 1184, 1148, 1070, 1053, 1031, 1014, 985, 906, 862, 844, 802, 768, 737, 710, 657 cm⁻¹. The Raman spectrum of the product (FIG. II) was recorded with the sample in an NMR tube using a Nicolet 960 E.S.P. FT-Raman spectrometer, at 4 cm⁻¹ resolution with excitation from a Nd:V04 laser (1064 nm) with a power output of 400 mW. Bands were observed at: 3067, 2997, 2926, 2884, 2860, 1747, 1611, 1588, 1545, 1445, 1382, 1360, 1315, 1287, 1240, 1213, 1185, 1070, 1016, 986, 917, 826, 769, 740, 712, 659, 636, 620, 605, 506, 470, 405, 332, 303, 134, 99 cm⁻¹.

The XRPD pattern of the product (FIG. III) was recorded using the following acquisition conditions: Tube anode: Cu, Generator tension: 40 kV, Generator current: 40 mA, Start angle: 2.0 °2θ, End angle: 35.0 °2θ, Step size: 0.02 °2θ, Time per step: 2.5 seconds. Characteristic XRPD angles and relative intensities are recorded in Table I.

TABLE 1

| Angle | Intensity |
|---|---|
| 2-Theta ° | % |
| 10.0 | 2.9 |
| 11.7 | 2.7 |
| 12.4 | 0.8 |
| 13.2 | 8.9 |
| 13.4 | 9.6 |
| 13.8 | 1.1 |
| 14.4 | 1.8 |
| 14.8 | 5.6 |
| 15.9 | 7.4 |
| 16.3 | 23.5 |
| 17.1 | 17.2 |
| 17.6 | 15.5 |
| 18.1 | 21.1 |
| 19.4 | 15.1 |
| 19.4 | 15.1 |
| 20.3 | 6.8 |
| 20.7 | 2.4 |
| 21.3 | 7.3 |
| 22.1 | 36.3 |
| 22.5 | 20.8 |
| 22.8 | 3 |
| 23.4 | 100 |
| 23.7 | 18 |
| 24.0 | 19.7 |
| 24.5 | 18.1 |
| 24.9 | 25.2 |
| 25.7 | 10.6 |
| 26.3 | 12 |
| 26.8 | 11.8 |
| 27.0 | 15.8 |
| 27.3 | 6.6 |
| 27.8 | 15.4 |
| 28.2 | 5 |
| 29.2 | 12.4 |
| 29.4 | 6.8 |
| 29.9 | 5.9 |
| 30.4 | 11.5 |
| 30.7 | 21.9 |
| 31.1 | 2.9 |
| 31.8 | 7.2 |
| 32.2 | 8.3 |
| 32.3 | 8.8 |
| 32.5 | 11.9 |
| 33.0 | 7.3 |
| 33.9 | 7 |
| 34.3 | 9 |
| 34.7 | 5.5 |

1. A compound 5-[4-[2-(N-methyl-N-(2-pyridyl)amino)ethoxy]benzyl]thiazolidine-2,4-dione hydrobromide or a solvate thereof.

2. A compound according to claim 1, characterised in that it provides two or more of:
(i) an infrared spectrum substantially in accordance with FIG. I;
(ii) a Raman spectrum substantially in accordance with FIG. II;
(iii) an X-Ray powder diffraction pattern (XRPD) substantially in accordance with Table I or FIG. III;
(iv) a solid-state ¹³C NMR spectrum substantially in accordance with FIG. IV; and
(v) a melting point within the range of from 175 to 185° C., especially 180 to 185° C., for example 181° C.

3. A compound according to claim 1 or claim 2, in purified form.

4. A compound according to any one of claims 1 to 3, in a solid dosage form.

5. A compound according to any one of claims 1 to 3, in a pharmaceutically acceptable form capable of being milled.

6. A compound according to any one of claims 1 to 3, in a pharmaceutically acceptable form having good flow properties.

7. A process for preparing the 5-[4-[2-(N-methyl-N-(2-pyridyl)amino)ethoxy]benzyl]thiazolidine-2,4-dione hydrobromide or a solvate thereof, characterised in that 5-[4-[2-(N-methyl-N-(2-pyridyl)amino)ethoxy]benzyl]thiazolidine-2,4-dione or a salt thereof is reacted with a source of hydrogen bromide and thereafter, if required, a solvate of the Hydrobromide is prepared; and the Hydrobromide or solvate thereof is recovered.

8. A pharmaceutical composition comprising 5-[4-[2-(N-methyl-N-(2-pyridyl)amino)ethoxy]benzyl]thiazolidine-2,4-dione hydrobromide or a solvate thereof, and a pharmaceutically acceptable carrier therefor.

9. A compound 5-[4-[2-(N-methyl-N-(2-pyridyl)amino)ethoxy]benzyl]thiazolidine-2,4-dione hydrobromide or a solvate thereof for use as an active therapeutic substance.

10. A use of 5-[4-[2-(N-methyl-N-(2-pyridyl)amino)ethoxy]benzyl]thiazolidine-2,4-dione hydrobromide or a solvate thereof, for the manufacture of a medicament for the S treatment and/or prophylaxis of diabetes mellitus, conditions associated with diabetes mellitus and certain complications thereof.
